(19) United States
(12) Patent Application Publication
(10) Pub. No.: US 2018/0344696 A1
(43) Pub. Date: Dec. 6, 2018

(54) SYNERGISTIC COMBINATION OF PYRROLIDONE CARBOXYLIC ACID AND/OR SALTS THEREOF AND HYALURONIC ACID AND/OR SALTS THEREOF, FOR USE IN THE TREATMENT AND/OR PREVENTION OF DRYNESS AND IRRITATION OF THE MUCOSAE, AND RELATED PHARMACEUTICAL FORMULATIONS

(71) Applicant: LABORATORI BALDACCI S.P.A., Pisa (IT)

(72) Inventor: Massimo BALDACCI, Pisa (IT)

(21) Appl. No.: 15/777,154

(22) PCT Filed: Dec. 5, 2016
(86) PCT No.: PCT/IB2016/057351

§ 371 Date: May 17, 2018

(30) Foreign Application Priority Data
Dec. 11, 2015 (IT) 102015000082426

(51) Int. Cl. A61K 31/4015; A61K 31/728; A61K 9/00; A61K 9/06; A61P 15/02; A61P 27/16; A61K 47/32; A61K 47/34; A61K 47/24; A61K 47/10; A61K 47/36; A61K 47/26; A61K 47/38; A61K 47/02

(52) U.S. Cl. CPC A61K 31/4015; A61K 47/02; A61K 9/0043; A61K 9/0034; A61K 9/06; A61P 15/02; A61P 27/16; A61K 47/32; A61K 47/34; A61K 47/24; A61K 47/10; A61K 47/36; A61K 47/26; A61K 47/38; A61K 31/728

(57) ABSTRACT

The object of the present invention is the synergistic combination of pyrrolidone carboxylic acid (PCA) and/or pharmaceutically acceptable salts thereof and hyaluronic acid and/or pharmaceutically acceptable salts thereof, for use in the treatment and/or prevention of dryness and irritation of the mucosae.

A further object of the present invention are pharmaceutical compositions comprising the synergistic combination of pyrrolidone carboxylic acid (PCA) and/or pharmaceutically acceptable salts thereof and hyaluronic acid and/or pharmaceutically acceptable salts thereof, and at least one physiologically acceptable excipient, and the use of such compositions in the treatment and/or prevention of dryness and irritation of the mucosae.

The object of the present invention is the synergistic combination of pyrrolidone carboxylic acid (PCA) and/or pharmaceutically acceptable salts thereof and hyaluronic acid and/or pharmaceutically acceptable salts thereof, for use in the treatment and/or prevention of dryness and irritation of the mucosae.

A further object of the present invention are pharmaceutical compositions comprising the synergistic combination of pyrrolidone carboxylic acid (PCA) and/or pharmaceutically acceptable salts thereof and hyaluronic acid and/or pharmaceutically acceptable salts thereof, and at least one physiologically acceptable excipient, and the use of such compositions in the treatment and/or prevention of dryness and irritation of the mucosae.

### STATE OF THE ART

The mucosa (mucous membrane, or tunica mucosa) is the tissue portion in direct contact with the lumen of animal hollow organs that are in communication with the external environment, such as the digestive tract, the respiratory tract, or mucous membranes of the genital apparatus.

It is formed by the superposition of four layers, namely i) a coating epithelium, which faces outwards, ii) a basal lamina, which forms the connection between the epithelial layer and the connective layer, and which is formed by lamina densa and lamina rare (depending on the protein composition), and from a fibroreticular lamina of connective origin; iii) an intermediate lamina or tunica propria of loose connective tissue in twisted bundles; and iv) a muscolaris mucosae, only present, however, in the organs of the digestive tract, starting from the esophagus, and consisting of a layer of smooth muscle fibrocells, which continues with the submucosa.

The mucosa is not waterproofed by keratin, but it is protected by mucus, rich in mucopolysaccharides. The mucus is in fact a viscous colloid, formed by the muciparous glands of several tissues in the body, which is produced in the folds of mucous membranes of organs and which generally contains antiseptic enzymes. It is made of glycosylated proteins and salts dissolved in water.

The mucus performs several important functions in the body.

In the respiratory system, the nasal cavities, which form integral part of the upper respiratory tract together with the pharynx, are covered by 140 to 160 cm² of mucosa. In turn, the latter is covered by a tissue containing numerous mucus-secreting cells (or nasal secretions), whose function is to humidify the air breathed, to capture bacteria and dust, preventing their entry into the body, particularly in the nose. The nasal mucosa is, in fact, crossed by nerve fibers that make it sensitive, fibers that are responsible, for example, of the sneezing reflex which rejects foreign bodies, thus preventing their entry.

A portion of the nasal cavity is lined by olfactory epithelium which allows to chemically analyze the air breathed. The second function of the nasal cavities is to heat the inhaled air. This is possible in the various spaces created by bone structures inside the nose, which are covered with a highly vascularized mucosa (rich in blood vessels).

Passing through the upper respiratory tract, the air must also become saturated with water vapor to avoid irritating the larynx, the lower respiratory tract, and, further down, the alveolar spaces (where gas exchange is produced). This humidification is made possible thanks to nasal secretions, and to the lacrimal fluid, that provide the necessary water vapor. This process is one of the main advantages of nasal breathing compared to the buccal one; the latter has a tendency to irritate the bronchi due to the fact that the inhaled air remains dry.

The mucous lining of the nasal cavities also has a purification function, which consists in holding the dust and other foreign bodies. The cilia movements reject them in the direction of the nostrils or pharynx.

The nasal cavities and the paranasal sinuses also act as a sounding board, and affect the timbre of the voice. Finally, these spaces filled with air are good thermal insulators. Keeping in mind all these functions, it is easy to understand why lesions of the mucosa may have unpleasant consequences.

In case of a cold, a congestion (swelling) of the mucosa is observed. Since the connective tissue of the nasal mucosa is crossed by numerous blood vessels, their expansion due to cold causes a rapid swelling of the mucosa as well as an excessive production of more or less fluid mucus. Nasal breathing becomes therefore difficult, and the buccal one causes rapid drying and irritation of the larynx.

Even the nasal mucosa may become dry: in this case we speak of dry rhinitis. This disease is characterized by the formation of crusts in the nose, itching, sneezing, and breathing difficulties. Since the production of mucus is insufficient, the normal process of air humidification and purification may not take place. This phenomenon causes irritation of the larynx and bronchi, which become more vulnerable and become infected more easily. Dry rhinitis may become chronic

There are several possible reasons: very dry climate, excessive heating, air conditioning, smoking, air pollution, or a dusty environment. Furthermore, in menopausal women, a “natural” drying up of the mucosae in general, particularly the nasal one, is observed. Some drugs, such as nasal decongestants, may also cause a dry rhinitis.

To avoid complications (lower respiratory tract irritation, nosebleeds, or even perforation of the nasal septum), the desiccation of the nasal mucosa has to be avoided or remidied.

Besides avoidance of factors that may irritate it, it is possible to hydrate the nasal mucosa by rinsing the nasal cavities with physiological serum. The application of a nasal cream, or a spray is, however, more pleasant and more practical. In the market different products, generally based on sodium chloride (kitchen salt) at physiological concentration (or isotonic), i.e. equivalent to that which normally occurs in the body, may be found. This is essential to avoid irritation of the mucosa.

Certain products also contain dexpanthenol, an alcohol derivative of pantothenic acid, or vitamin B5. Finally, a topical nasal treatment containing Dead Sea salt instead of sodium chloride is also available on the market.

However, these remedies, while having an immediate hydrating action, do not provide control of dry rhinitis in the long run.

In the digestive tract, mucus is used as a lubricant for the passage of food through the intestines, and also serves to make the internal surfaces flexible. In the stomach, mucus is of fundamental importance because it acts as an additional protection to the gastric mucosa from digestive acids (HCl) in the gastric juice.

In the female reproductive system, finally, mucus helps prevent infections, helps sexual intercourse, and prevents dryness of mucosae.

The dryness of the vaginal mucosa is a frequent problem in the pre- and post-menopause, although inadequate vaginal lubrication may occur at any age. Vaginal dryness can be a symptom of vaginal atrophy (atrophic vaginitis), that is thickening and inflammation of the vaginal mucosa due to a decrease in estrogens. Along with vaginal dryness, one may suffer from itching and burning around the opening and the bottom of the vagina, which make sexual intercourse difficult.

However, hormonal changes due to menopause, pregnancy, and lactation may affect this process.

The decrease in estrogen levels is, in fact, the main cause of vaginal dryness.

The levels of estrogens may decrease for many different reasons; besides the already mentioned menopause or perimenopause, pregnancy, or breast-feeding, there may be side effects of cancer therapy (radiation therapy, hormonal therapy, and chemotherapy), ovary removal surgery, immune disorders, cigarette smoking, or drugs. For example, anti-allergic and anti-influenza drugs, as well as some antidepressants, may decrease the hydration of different parts of the body, including the vagina. Also antiestrogens, for example, those used to treat breast cancer, may cause vaginal dryness.

Finally, vaginal dryness may also be caused by Sjogren's syndrome, namely an autoimmune disease that causes dry eyes and mouth, but also vaginal dryness. Vaginal dryness may be accompanied by very unpleasant symptoms, such as itching, burning, pain, pain or mild bleeding during intercourse, frequent urge, or urgency to urinate.

In addition to the possible use of estrogens, to remedy vaginal dryness water-based lubricants, for use before sexual intercourse, or hydrating products which mimic natural secretions, are sold. However, even in this case, most of the commercially available products are limited to an action which is effective but short in time.

The need is therefore felt for a treatment and/or effective prevention of dryness and irritation of the mucosae, particularly the nasal and/or vaginal ones, able to guarantee an improved hydration both immediate, and that persists for a long time.

Pyrrolidone carboxylic acid (PCA), depicted in the formula reported below, is a cyclic organic compound, also known as pyroglutamic acid. (I).

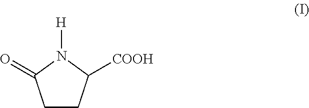

In the literature, the action of PCA as a vector to promote an improved gastrointestinal adsorption of drugs has long been described (Barel et al. Handbook of Cosmetic Science and Technology, Third Edition, pp. 357-370, 2009; Smith et al. Percutaneous Penetration Enhancers, CRC Press, p. 214, 1995). The emollient and moisturizing properties of the product are also known, when it is used in various cosmetic compositions for skin and hair care.

PCA is added in the European databank of Cosmetic Ingredients as a humectant and moisturizer: ec.Europa.eu/consumers/cosmetics/cosing.

No toxic and/or harmful effects have ever been found, neither when this compound has been used topically nor when administered, even in high doses, in humans and laboratory animals.

Hyaluronic acid of formula (II) is one of the primary components of connective tissues in humans and other mammals. It provides the skin with its peculiar resistance properties and shape retention. Its absence causes a weakening of the skin, promoting the formation of wrinkles and blemishes. Its concentration in body tissues tends to decrease with age. It is sold on the market, for cosmetic use, under the name of sodium hyaluronate because it is treated in order to adjust the pH.

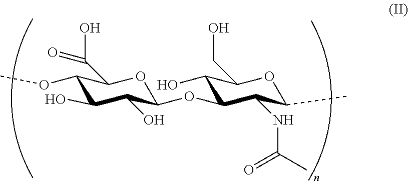

Chemically, hyaluronic acid may be defined as a sulfide-free glycosaminoglycan lacking of a protein core, with unbranched polysaccharide chain produced by the condensation of thousands of disaccharide units formed, in turn, by residues of glucuronic acid and N-acetylglucosamine, linked together, alternatively, by 1→4 and β1→3 glycosidic bonds, as well as by intramolecular hydrogen bonds, that stabilize the conformations. At physiological pH, the carboxyl groups of glucuronic units are ionized, conferring high polarity to the molecule of hyaluronate, and accordingly high solubility in water. Thanks to this property, hyaluronate is able to complex with many water molecules reaching a high degree of hydration. Hyaluronates are macromolecules with a mass greater than 1000 kDa, that give rise to clear high viscosity solutions.

Injections of hyaluronic acid are used in conjunction with injections of collagen proteins in surgery and cosmetic dermatology to remove wrinkles, and prevent aging of the skin. In otologic surgery, hyaluronic acid is used to regenerate perforated tympanic membranes, in ophthalmic surgery for the production of artificial tears and interventions on the vitreous body of the eye, in arthrology as an antiphlogistic lubricant, and to preserve the synovial fluid of the joints. It is also used against inflammation and ulcerative lesions of the mouth (mouth ulcers, stomatitis etc.), particularly those resulting from chemotherapy and radiotherapy, immediately reducing pain and promoting healing. The commercial product is in the form of a gel, spray, and mouthwash. The gel or spray is employed directly on ulcerated areas, with persistent pain it can also be used several times a day without any contraindications or side effects, except for specific allergies.

### Definitions

Unless otherwise defined, all terms of the art, notations and other scientific terms used herein are intended to have the meanings commonly understood by those skilled in the art to which this description belongs. In some cases, terms with meanings that are commonly understood are defined herein for clarity and/or ready reference; therefore, the inclusion of such definitions herein should not be interpreted as being representative of a substantial difference with respect to what is generally understood in the art.

The term “pharmaceutically acceptable salts or derivatives” refers to those salts or derivatives which possess the biological effectiveness and properties of the salified compound, and that do not produce adverse reactions when administered to a mammal, preferably a human being. The pharmaceutically acceptable salts may be inorganic or organic salts; examples of pharmaceutically acceptable salts include, but are not limited to: carbonate, hydrochloride, hydrobromide, sulfate, hydrogen sulfate, citrate, maleate, fumarate, trifluoroacetate, 2-naphthalenesulfonate, and para-toluenesulfonate. Additional information on pharmaceutically acceptable salts may be found in Handbook of pharmaceutical salts, P. Stahl, C. Wermuth, WILEY-VCH, 127-133, 2008, incorporated herein by reference.

The term “physiologically acceptable excipient” refers to a substance devoid of any pharmacological effect of its own, and that does not produce adverse reactions when administered to a mammal, preferably a human being. Physiologically acceptable excipients are well known in the art and are described, for example, in Handbook of Pharmaceutical Excipients, sixth edition 2009, incorporated herein by reference.

The term “simultaneous, separate or sequential use” refers to the simultaneous administration of the first and the second compound, or in such a way that the two compounds will act on the mucosa of the patient at the same time, or to the administration of a compound after the other compound in such a way to provide a therapeutic effect. In some embodiments, a compound is administered to a patient for a period of time, followed by the administration of the other compound.

The terms “comprising”, “having”, “including” and “containing” are to be intended as open terms (i.e., meaning “comprising, but not limited to”), and are to be considered as a support also for terms such as “consist essentially of”, “consisting essentially of”, or “consisting of”.

The term “q.s.” refers to the amount needed to reach the indicated volume.

### DESCRIPTION OF THE INVENTION

It has now surprisingly been found that the synergistic combination of pyrrolidone carboxylic acid (PCA) and/or pharmaceutically acceptable salts thereof and hyaluronic acid and/or pharmaceutically acceptable salts thereof is particularly effective for use in the treatment and/or prevention of dryness and irritation of the mucosae, in particular the nasal and vaginal mucosa.

An object of the present invention is, therefore, pyrrolidone carboxylic acid and/or pharmaceutically acceptable salts thereof in combination with hyaluronic acid and/or pharmaceutically acceptable salts thereof for simultaneous, separated or sequential use in the treatment and/or prevention of dryness and irritation of the mucosae, in particular the nasal and vaginal mucosa.

According to a preferred aspect, the combination of pyrrolidone carboxylic acid and/or pharmaceutically acceptable salts thereof and hyaluronic acid and/or pharmaceutically acceptable salts thereof for use in the treatment and/or prevention of dryness and irritation of the mucosae, in particular the nasal and vaginal mucosa, is characterised by being administered as a pharmaceutical formulation comprising pyrrolidone carboxylic acid and/or pharmaceutically acceptable salts thereof in an amount comprised between 0.05% and 2% by weight, based on the total weight of the formulation.

According to a further preferred aspect, the combination of pyrrolidone carboxylic acid and/or pharmaceutically acceptable salts thereof and hyaluronic acid and/or pharmaceutically acceptable salts thereof for use in the treatment and/or prevention of dryness and irritation of the mucosae, in particular the nasal and vaginal mucosa, is characterised by being administered as a pharmaceutical formulation comprising hyaluronic acid and/or pharmaceutically acceptable salts thereof, preferably sodium hyaluronate, in an amount comprised between 0.05% and 1% by weight, based on the total weight of the formulation. Particularly preferred percentages of hyaluronic acid and/or pharmaceutically acceptable salts thereof, preferably sodium hyaluronate, are 0.05%, 0.1%, 0.5%, and 1%, where each percentage is a percentage by weight, based on the total weight of the formulation.

According to a further preferred aspect, the combination of pyrrolidone carboxylic acid and/or pharmaceutically acceptable salts thereof and hyaluronic acid and/or pharmaceutically acceptable salts thereof for use in the treatment and/or prevention of dryness and irritation of the mucosae, in particular the nasal and vaginal mucosa, is characterised by being administered as a pharmaceutical formulation having a pH comprised between 3 and 7.5, preferably between 4.5 and 6.5, and even more preferably of between 4 and 5.

The preferred synergic combination of pyrrolidone carboxylic acid and/or pharmaceutically acceptable salts thereof and hyaluronic acid and/or pharmaceutically acceptable salts thereof for use in the treatment and/or prevention of dryness and irritation of the mucosae, in particular the nasal and vaginal mucosa, is characterised by being administered as a pharmaceutical formulation comprising pyrrolidone carboxylic acid and/or pharmaceutically acceptable salts thereof in an amount comprised between 0.05% and 2% by weight, based on the total weight of the formulation, comprising hyaluronic acid and/or pharmaceutically acceptable salts thereof, preferably sodium hyaluronate, in an amount comprised between 0.05% and 1% by weight, based on the total weight of the formulation, comprising at least one physiologically acceptable excipient, and wherein said formulation has a pH comprised between 3 and 7.5, preferably between 4.5 and 6.5, and even more preferably of between 4 and 5.

According to a further preferred aspect, the combination of pyrrolidone carboxylic acid and/or pharmaceutically acceptable salts thereof and hyaluronic acid and/or pharmaceutically acceptable salts thereof for use in the treatment and/or prevention of dryness and irritation of the mucosae, is not used with additional active ingredients. That is, pyrrolidone carboxylic acid and/or pharmaceutically acceptable salts thereof and hyaluronic acid and/or pharmaceutically acceptable salts thereof are the only active ingredients present in the synergic combination of the invention, and are not used together with additional active ingredients for use in the treatment of the dryness of the mucosae.

A further object of the present invention are pharmaceutical compositions comprising the synergic combination of pyrrolidone carboxylic acid and/or pharmaceutically acceptable salts thereof and hyaluronic acid and/or pharmaceutically acceptable salts thereof, and at least a physiologically acceptable excipient, and use of such compositions in the treatment and/or prevention of dryness and irritation of the mucosae, in particular the nasal and vaginal mucosa.

According to a preferred aspect, the compositions of the invention comprise the synergic combination of pyrrolidone carboxylic acid (PCA) and/or pharmaceutically acceptable salts thereof and hyaluronic acid and/or pharmaceutically acceptable salts thereof, as the only active ingredients, preferably each in the percentages by weight, based on the total weight of the composition, listed above, together with at least a physiologically acceptable excipient.

According to an aspect of the invention, said at least one physiologically acceptable excipient is selected from preservatives, antioxidants, buffering agents, moisturizers (such as glycerine or sorbitol, preferably at 70%), stabilizers, viscosity agents (silicone vehicles such dimethiconol and dimethicone (Dow Corning TI3011), silicone elastomers (Dow Corning TI3021)), gelling polyacrylates (carbomer), surfactants, water, preferably purified water, aqueous vehicles, oleaginous vehicles, humectants, gelling agents, or mixtures thereof.

According to a preferred aspect, a buffering system (such as, sodium hydroxide or phosphate buffer) and/or a preservative may be present. Examples of preferred preservatives are parabens in phenoxyethanol, phenoxyethanol in ethylhexylglycerin (Euxyl PE9010), imidazolidinyl urea, sodium dehydroacetate, sodium benzoate, potassium sorbate, benzyl alcohol or dehydroacetic acid in water, or mixtures thereof.

According to yet a further aspect of the invention, adhesion promoters (mucoadhesive substances) may be present. Preferred adhesion promoters are chitosan, cellulose derivatives, such as carboxymethylcellulose, ethylcellulose, hydroxyethylcellulose, hydroxypropylcellulose, hydroxypropylmethylcellulose, or a mixture thereof, polyvinyl alcohol, xanthan gum, alginate, preferably sodium alginate, or mixtures thereof.

According to an aspect of the invention, the pharmaceutical formulation of the invention is in a liquid or semi-solid form.

According to a further preferred aspect, the pharmaceutical formulations of the invention are administered topically.

According to a preferred aspect, the formulation of the invention is in the form of a solution (more preferably an aqueous solution), suspension, cream, ointment, gel, ovules, pessaries, vaginal tablets or spray. Most preferred is the gel formulation.

According to an aspect of the invention, the pharmaceutical formulation has a pH comprised between 3 and 7.5, preferably between 4.5 and 6.5, and even more preferably of between 4 and 5, so as to be physiologically applicable to the mucosa level without causing any side effects. The above-mentioned pH is obtained by addition of suitable buffers, such as phosphate buffer, or by addition of sodium hydroxide.

According to a preferred aspect, pyrrolidone carboxylic acid (PCA) and/or pharmaceutically acceptable salts thereof is contained in the pharmaceutical formulations of the invention in an amount comprised between 0.05% and 2% by weight, based on the total weight of the formulation.

According to a further preferred aspect, hyaluronic acid and/or pharmaceutically acceptable salts thereof, particularly the sodium salt, i.e. sodium hyaluronate, is contained in the pharmaceutical formulations of the invention in an amount comprised between 0.05% and 1% by weight, based on the total weight of the formulation.

Particularly preferred percentage values for hyaluronic acid and/or pharmaceutically acceptable salts thereof, preferably sodium hyaluronate, are 0.05%, 0.1%, 0.5%, and 1%, where each percentage is a percentage by weight,based on the total weight of the formulation.

The preferred pharmaceutical formulation of pyrrolidone carboxylic acid and/or pharmaceutically acceptable salts thereof and hyaluronic acid and/or pharmaceutically acceptable salts thereof for use in the treatment and/or prevention of dryness and irritation of the mucosae, particularly the nasal and/or vaginal mucosa, comprises pyrrolidone carboxylic acid and/or pharmaceutically acceptable salts thereof in an amount comprised between 0.05% and 2% by weight, based on the total weight of the formulation, comprises hyaluronic acid and/or pharmaceutically acceptable salts thereof, preferably sodium hyaluronate, in an amount comprised between 0.05% and 1% by weight, based on the total weight of the formulation, comprises at least one physiologically acceptable excipient, and said formulation has a pH comprised between 3 and 7.5, preferably between 4.5 and 6.5, and even more preferably of between 4 and 5.

According to a further preferred aspect, the weight ratio between pyrrolidone carboxylic acid and/or pharmaceutically acceptable salts thereof and hyaluronic acid and/or pharmaceutically acceptable salts thereof in the combination of the invention, or in the composition comprising it, is 40.

Preferably, pyrrolidone carboxylic acid and/or pharmaceutically acceptable salts thereof and hyaluronic acid and/or pharmaceutically acceptable salts thereof, are the only active ingredients of said pharmaceutical formulation.

Even more preferably, said pharmaceutical composition is for topical use, and it is in the form of a gel, particularly a nasal and/or vaginal gel.

Preferably, the molecular weight of the hyaluronic acid used is comprised in the range of 1-1.4 MDa.

Without being bound by a specific scientific theory, it has been seen that the immediate hydrating effect of hyaluronic acid is followed by the long term hydration effect of the mucosae due to pyrrolidone carboxylic acid.

In this way, it is possible to solve the problem of mucosal dryness, especially at the nasal or vaginal level, helping to prevent the recurrence of the problem.

The pyrrolidone carboxylic acid (PCA) of the invention has shown a surprising effect in improving and maximizing hydration of the mucosae, particularly of the nasal and vaginal mucosae, especially in terms of duration of the hydration itself, and as an effect of water retention within the mucosal tissue.

The pyrrolidone carboxylic acid (PCA) remains, in fact, at the at the level of the mucosal tissue for a much longer time than the compounds currently used in the treatment of mucosal dryness. PCA remains in the mucosae for a time longer than 4 hours, after its topical application, when normally a product indicated for the treatment of dryness of the mucosae remains for no longer than 1 hour.

It is therefore obvious that a synergistic formulation containing pyrrolidone carboxylic acid (PCA) and hyaluronic acid may be applied on the mucosae that require hydration for a number of times significantly lower, compared to the formulations presently known. As an indication, the known products are applied in case of dryness of the mucosae from 6 to 8 times a day, while a formulation containing PCA and hyaluronic acid according to the present invention would be applied from 1 to 3 times a day, improving the patient or the interested subject compliance.

The reduction of the repetition of the administration in the case of mucosal dryness is of great importance, in order to improve the treatment, and the impact of the same on the life of the subject or patient. A result of the improved hydration is also the refreshing ability generated by the formulation upon administration.

The addition of hyaluronic acid has surprisingly generated a synergy in terms of hydrating effect. In fact, the hyaluronic acid, already a moisturizer with immediate effectiveness per se, also functions as a co-adhesive, and binds to the surface of the mucosae retaining PCA that exerts the humectant and hydration effect, thus prolonging even more the hydrating effect of PCA.

4 hours after a topical application of the formulations of the invention, the analysis of mucosal tissues has allowed the identification of approximately 80% of the administered amount of PCA.

Moreover, all the formulations tested have proved completely free of side effects, even after prolonged treatment for at least 15 days.

The following examples are intended to better illustrate the present invention without limiting it in any way

### EXAMPLES

### Example 1: Gel for Nasal Mucosa

| | % by weight, based on the |
|---|---|
| | total weight of the |
| Nasal gel | formulation |
| Pyrrolidone carboxylic acid (PCA) | 1.50 |
| Hyaluronic acid | 0.05 |
| Mixture of sodium polyacrylate, | 5.50 |
| dimethicone, cyclopentasiloxane, | |
| trideceth-6 and | |
| PEG/PPG 18/18 dimethicone | |
| (Dow Corning RM2051) | |
| Mixture of high molecular weight | 14.50 |
| dimethiconol and non-volatile | |
| dimethicone | |
| (Dow Corning TI3011) | |
| Silicone elastomer dispersed in | 30.00 |
| dimethicone | |
| (Dow Corning TI3021) | |
| Phenoxyethanol in ethylhexylglycerin | 1.00 |
| (Euxyl PE9010) | |
| Water | q.s. to 100 |

The two silicone components are mixed with an aqueous system, which contains PCA and hyaluronic acid, thanks to the presence of Dow Corning RM2051 product, which is a thickener and emulsifier mixture based on silicones and polyacrylates, specifically designed to stabilize water-silicone emulsions, and equipped with film-forming capability able to further stabilize the gel layer on the nasal mucosa.

The presence of an aqueous phase, in addition to allowing the dissolution of the two synergistic active ingredients, PCA and hyaluronic acid, makes the product fresh, easy to apply and pleasant to the touch.

### Example 2: Gel for Nasal Mucosa

| | % by weight, based on the total |
|---|---|
| Nasal gel | weight of the formulation |
| Pyrrolidone carboxylic acid (PCA) | 1.00 |
| Sodium hyaluronate | 0.05 |
| Chitosan | 0.20 |
| Polyacrylate gelling agents (Carbomer) | 0.40 |
| Parabens in phenoxyethanol | 0.70 |
| Purified water | q.s. to 100 |

### Example 3: Gel for Nasal Mucosa

| | % by weight, based on the total |
|---|---|
| Nasal gel | weight of the formulation |
| Pyrrolidone carboxylic acid (PCA) | 0.80 |
| Sodium hyaluronate | 0.05 |
| Glycerin | 3.00 |
| Sorbitol solution 70% | 1.00 |
| Polyacrylate gelling agents (Carbomer) | 0.40 |
| Sodium hydroxide | q.s. to pH 6.0 |
| Parabens in phenoxyethanol | 0.70 |
| Purified water | q.s. to 100 |

### Example 4: Gel for Vaginal Mucosa

| | % by weight, based on the total |
|---|---|
| Vaginal gel | weight of the formulation |
| Pyrrolidone carboxylic acid (PCA) | 1.50 |
| Sodium hyaluronate | 0.05 |
| Glycerin | 3.00 |
| Sorbitol solution 70% | 1.00 |
| Crosslinked polyacrylic acid (Polycarbophil) | 0.80 |
| Hydroxyethylcellulose | 0.80 |
| Sodium hydroxide | q.s. to pH 5.0 |
| Parabens in phenoxyethanol | 0.70 |
| Purified water | q.s. to 100 |

### Example 5: Gel for Vaginal Mucosa

| | % by weight, based on the total |
|---|---|
| Vaginal gel | weight of the formulation |
| Pyrrolidone carboxylic acid (PCA) | 2.00 |
| Sodium hyaluronate | 0.05 |
| Glycerin | 3.00 |
| Sorbitol solution 70% | 1.00 |
| Hydroxyethylcellulose | 1.30 |
| Parabens in phenoxyethanol | 0.70 |
| Purified water | q.s. to 100 |

### Example 6: Determination of PCA Permanence on the Nasal Mucosa

The determination of pyrrolidone carboxylic acid (PCA) permanence on the nasal mucosa was performed on 6 adult male subjects using the gel formulation for the nasal mucosa containing 1% of PCA and 0.05% of hyaluronic acid, i.e. the formulation of Example 2 of a gel for nasal mucosa. 100 mg of gel were applied on the nasal mucosa of each subject.

The samples were taken at various time points after administration, in order to accurately collect all the material present on the mucosa.

After restoring to a certain volume of water (20 ml), the suspension obtained was filtered, and then 20 μl were injected into a chromatograph for quantitative analysis. The results are reported in Table 1 below.

A Waters liquid chromatograph with the following parameters was used:
Column: μBondapack C18
Mobile phase: 0.1 M phosphoric acid at pH 3 with sodium hydroxide.
Flow: 1.5 ml/min
Detector: Spectrophotometer at 210 nm

TABLE 1
PCA percentage (%) present in the nasal mucosa at
different time points after application

| Time after | % of recovery based |
|---|---|
| administration | on the standard |
| T = 0 | 95% n = 1 |
| T = 60 | 80% n = 2 |
| T = 120 | 78% n = 2 |
| T = 180 | 70% n = 3 |
| T = 240 | 55% n = 2 |

n = number of tests carried out

The experimental results obtained show that PCA is present on the nasal mucosa at least up to 4 hours after application of the gel formulation from Example 2 on the nasal mucosa, with a percentage of unchanged product of 55% based on the standard.

### Example 7: Determination of Hygroscopicity of the Combination of Pyrrolidone Carboxylic Acid (PCA) and Hyaluronic Acid (HA)

The determination of hygroscopicity of the combination of PCA and hyaluronic acid was performed, a test related to the hydration capability of a compound or mixture of compounds, to evaluate the synergism of the combination of PCA and hyaluronic acid for use in the treatment and/or prevention of dryness and irritation of the mucosae.

The hygroscopic test was performed according to the Farmacopea Ufficiale Italiana (FUI) XII: first, a glass container with an outer diameter of 50 mm and a height of 15 mm with its lid (m1) was weighed, then 5 g of completely dried sample were transferred into the container (m2); the container, without lid, was then placed in a suitable desiccator containing an ammonium chloride or ammonium sulfate saturated solution, at a temperature of 25° C. for 24 hours. At the end of the procedure, the contained was closed with its lid and weighed again (m3). The percentage of mass increase was calculated using the following formula:

(m3−m2)/(m2−m1)×100

The result was interpreted as follows:
Deliquescent—absorption of sufficient water to form a liquid;
Highly hygroscopic—mass increase equal to or greater than 15%;
Hygroscopic—mass increase of less than 15%, and equal to or greater than 2%;
Slightly hygroscopic—mass increase of less than 2%, and equal to or greater than 0.2%.

The results are reported in Table 2 below:

TABLE 2
Hygroscopicity of PCA and HA combination at different ratios

| | | | Water | Water | |
|---|---|---|---|---|---|
| | | | adsorption | adsorption | |
| | | | (g) (average | (%) | |
| | Initial | Final | of the | (average of | |
| Sample | Weight | Weight | triplicate) | the triplicate) | Result |
| 100% PCA | 5.00 g | 5.05 ± 0.01 g | 0.05 g | 1.01% | slightly |
| | | | | | hygroscopic |
| 100% HA | 5.00 g | 5.30 ± 0.05 g | 0.30 g | 6.00% | hygroscopic |
| 99% PCA + | 5.00 g | 5.09 ± 0.02 g | 0.09 g | 1.90%* | slightly |
| 1% HA | | | | | hygroscopic |
| 97.5% PCA + | 5.00 g | 5.13 ± 0.05 g | 0.13 g | 2.55%* | hygroscopic |
| 2.5% HA | | | | | |
| 95% PCA + | 5.00 g | 5.13 ± 0.03 g | 0.13 g | 2.60%* | hygroscopic |
| 5% HA | | | | | |
| 90% PCA + | 5.00 g | 5.16 ± 0.03 g | 0.16 g | 3.24%** | hygroscopic |
| 10% HA | | | | | |
| 80% PCA + | 5.00 g | 5.32 ± 0.08 g | 0.32 g | 6.40%*** | hygroscopic |
| 20% HA | | | | | |

*p < 0.05 vs PCA
**p < 0.01 vs PCA
***p < 0.01 vs PCA

The association between PCA and hyaluronic acid was hygroscopic when the weight ratio of PCA:hyaluronic acid was 40.

The association between PCA and low concentrations of hyaluronic acid is synergistic, if it is considered that PCA per se is only slightly hygroscopic and that hyaluronic acid, when associated to PCA, increases its capability to adsorb water.

1-15. (canceled)

16. A method for treating and/or preventing dryness and irritation of the mucosae, comprising administering to a subject in need thereof a pharmaceutical formulation comprising (a) pyrrolidone carboxylic acid and/or pharmaceutically acceptable salts thereof in an amount between 0.05% and 2% by weight and (b) hyaluronic acid and/or pharmaceutically acceptable salts thereof in an amount between 0.05% and 1% by weight.

17. The method of claim 16, wherein the mucosae are selected from the group consisting of nasal mucosa and vaginal mucosa.

18. The method of claim 16, wherein the pharmaceutical formulation is administered from 1 to 3 times a day.

19. The method of claim 16, wherein the pharmaceutical formulation has a pH between 3 and 7.5.

20. The method of claim 16, wherein the pharmaceutical formulation comprises at least one physiologically acceptable excipient.

21. The method of claim 16, wherein pyrrolidone carboxylic acid and/or pharmaceutically acceptable salts thereof and hyaluronic acid and/or pharmaceutically acceptable salts are the only active ingredients in the pharmaceutical formulation.

22. The method of claim 16, wherein the pharmaceutical formulation is administered topically.

23. The method of claim 16, wherein the pharmaceutical formulation is in the form of a solution, a suspension, cream, ointment, gel, ovules, pessaries, vaginal tablet, or spray.

24. A method for treating and/or preventing dryness and irritation of the mucosae, comprising administering to a subject in need thereof a first pharmaceutical formulation comprising pyrrolidone carboxylic acid and/or pharmaceutically acceptable salts thereof in an amount between 0.05% and 2% by weight, and a second pharmaceutical formulation comprising hyaluronic acid and/or pharmaceutically acceptable salts thereof in an amount between 0.05% and 1% by weight.

25. The method of claim 24, wherein the first pharmaceutical formulation and the second pharmaceutical formulation are administered separately or sequentially.

26. The method of claim 24, wherein the mucosae are selected from the group consisting of nasal mucosa and vaginal mucosa.

27. The method of claim 24, wherein the first pharmaceutical formulation and the second pharmaceutical formulation are administered from 1 to 3 times a day.

28. The method of claim 24, wherein the first pharmaceutical formulation and the second pharmaceutical formulation comprise at least one physiologically acceptable excipient.

29. The method of claim 24, wherein the first pharmaceutical formulation and the second pharmaceutical formulation are administered topically.

30. The method of claim 24, wherein the first pharmaceutical formulation and the second pharmaceutical formulation are in the form of a solution, a suspension, cream, ointment, gel, ovules, pessaries, vaginal tablet, or spray.
